(19) United States
(12) Patent Application Publication
(10) Pub. No.: US 2019/0298565 A1
(43) Pub. Date: Oct. 3, 2019

(54) TEMPERATURE-ALTERING AND POSTURE ENHANCING VEST

(71) Applicant: Andrew C. Jacobs, Short Hills, NJ (US)

(72) Inventor: Andrew C. Jacobs, Short Hills, NJ (US)

(21) Appl. No.: 16/365,970

(22) Filed: Mar. 27, 2019

(30) Foreign Application Priority Data
Mar. 27, 2018 (CA) 2999454

(51) Int. Cl. A61F 5/02; A61F 7/02

(52) U.S. Cl. CPC A61F 5/02; A41D 1/04; A61F 7/02

(57) ABSTRACT

A garment, such as a vest, that may enable the selective warming or cooling of various portions of the wearer's body by, for example, using repositionable thermal packets removably affixed to the vest, and may enhance the wearer's posture by, for example, using of strategically placed straps or boning.

### FIELD OF THE INVENTION

This invention relates to the field of garments for selective heating or cooling parts of the body, and supporting and enhancing the wearer's posture.

### BACKGROUND OF THE INVENTION

The benefits of applying thermal packets to the body are well known. For example, thermal packets that generate heat may be used to help keep a person warm when participating in cold weather activities. They may also be applied to sore or injured muscles to relieve pain and accelerate healing. Cold thermal packs can be used to reduce inflammation or bring the temperature of the body down, among other uses. Additional benefits include help with body aches, Hot Flashes, swollen joints and blood clots.

Manually applying thermal packets to one's body using one's own hands has several disadvantages. First, manual application is limited to areas of the body that a person can reach. Second, even for reachable areas, it can be tiresome or awkward to hold a thermal packet in place for an extended period of time. Third, the heat or cold from the thermal packet will be transferred not only to the part of the body requiring a temperature change, but also the person's hands, which may be undesirable and uncomfortable. Fourth, even if a person employs both of their hands to apply thermal packets to their body, only a limited number of thermal packets can be manually applied at one time. Fifth, applying thermal packets to the body, either with one or two hands, further limits a persons' mobility and the activities in which they may participate while applying the packets.

Thermal packets may also be attached to the body using straps, wraps, adhesives, or other similar means. Using such attachment means, however, may restrict movement. Also, if straps or wraps are applied too tightly, they can impede circulation and potentially disrupt blood flow through the body part to which they are applied.

Garments that incorporate thermal packets are known in the art and are meant to address some of the aforementioned disadvantages. For example, U.S. Pat. No. 8,105,371, issued on Jan. 31, 2012 to Giocondo, discloses a vest having pairs of coupling strips to which envelopes holding thermal packets may be attached. U.S. Pat. No. 7,739,748, issued on Jun. 22, 2010 to SnapBac, LLC, discloses garments with inner and outer layers and space in between the layers for the insertion of thermal packets. U.S. Pat. No. 6,189,149, issued on Feb. 20, 2001 to Allen, discloses a vest with horizontally disposed pockets into which thermal packets may be inserted.

Although the prior art garments allow for the application of thermal packets to the body, they do not address the negative effects the garment can have on the wearer's posture. The thermal packets can be heavy. If the packets are only being used on the front of the garment, the weight can cause the wearer to hunch over. If the packets are only being used on one side of the garment, the body may lean in that direction. Attempting to realign one's body into an ideal position in the face of this additional weight can put unwanted strain on the wearer's back. This can be particularly troubling if the user is wearing the garment to assuage a back problem in the first place.

Thus, a need exists for a garment that incorporates thermal packets to allow for the selective heating or cooling of parts of the body, but also incorporates features for supporting and enhancing the wearer's posture.

### SUMMARY OF THE INVENTION

The present invention is direct to a garment, such as a vest, that serves one of or both of the following two purposes. First, it may enable the selective warming or cooling of various portions of the wearer's body by, for example, using repositionable thermal packets removably affixed to the vest. Second, the garment may enhance the wearer's posture by, for example, using of strategically placed straps or boning.

Straps around the belly and/or over the shoulders may reinforce the vest compression material at specifics areas that may be experiencing pain, and/or directly over various discs and muscles. Tightening the straps may also increase the effectiveness of cold and/or hot packs located between the straps and the user's body.

It is an object of the present invention to help offset the problems with carrying weight required by certain jobs, such as equipment carried by law enforcement officials such as a heavy holster and or body armor, reducing the effects of immediate and long term body deterioration.

It is a further objection of the present invention to enable people who cannot stand for extended period of time because of disc deterioration pain to remain upright longer and walk. The use of the garment according to the present invention may reduce overall back pain sufficient to allow people considering the use of Opioid Drugs or surgery delay medication or surgery temporarily or indefinitely. Wearing the garment and specialized “dancing” may also temporarily reduce or stop the effects of Sciatica.

### BRIEF DESCRIPTION OF THE DRAWINGS

A further understanding of the invention can be obtained by reference to embodiments set forth in the illustrations of the accompanying drawings. Although the illustrated embodiments are merely exemplary of systems, methods, and apparatuses for carrying out the invention, both the organization and method of operation of the invention, in general, together with further objectives and advantages thereof, may be more easily understood by reference to the drawings and the following description. Like reference numbers generally refer to like features (e.g., functionally similar and/or structurally similar elements).

The drawings are not necessarily depicted to scale; in some instances, various aspects of the subject matter disclosed herein may be shown exaggerated or enlarged in the drawings to facilitate an understanding of different features. Also, the drawings are not intended to limit the scope of this invention, which is set forth with particularity in the claims as appended hereto or as subsequently amended, but merely to clarify and exemplify the invention.

FIG. 1 depicts a front view of an exemplary embodiment of the invention;

FIG. 2 depicts a back view of the exemplary embodiment;

FIG. 3 depicts a view of the exemplary embodiment in an open position;

FIG. 4 depicts an alternate view of the exemplary embodiment in an open position.

FIGS. 5A and 5B depict a fastening band in accordance with the present invention.

### DETAILED DESCRIPTION OF THE INVENTION

The invention may be understood more readily by reference to the following detailed descriptions of embodiments of the invention. However, techniques, systems, and operating structures in accordance with the invention may be embodied in a wide variety of forms and modes, some of which may be quite different from those in the disclosed embodiments. Also, the features and elements disclosed herein may be combined to form various combinations without exclusivity, unless expressly stated otherwise. Consequently, the specific structural and functional details disclosed herein are merely representative. Yet, in that regard, they are deemed to afford the best embodiment for purposes of disclosure and to provide a basis for the claims herein, which define the scope of the invention. It must be noted that, as used in the specification and the appended claims, the singular forms “a”, “an”, and “the” include plural referents unless the context clearly indicates otherwise.

The present invention provides a temperature-altering and posture enhancing garment. The garment may be made from any fabric known in the art for use in creating clothing, including but not limited to a woven cotton, nylon, neoprene, or other fabric. The general purpose of the present invention, which will be described subsequently in greater detail, is to provide a new and improved garment for heating or cooling the body which has all the advantages of the prior art and none of the disadvantages.

The present invention is described below in the context of a garment intended to be worn on the upper body, such as a vest, jacket, shirt, or pullover. However, the present invention may also be applied to garments worn on other parts of the body, such as shorts or pants.

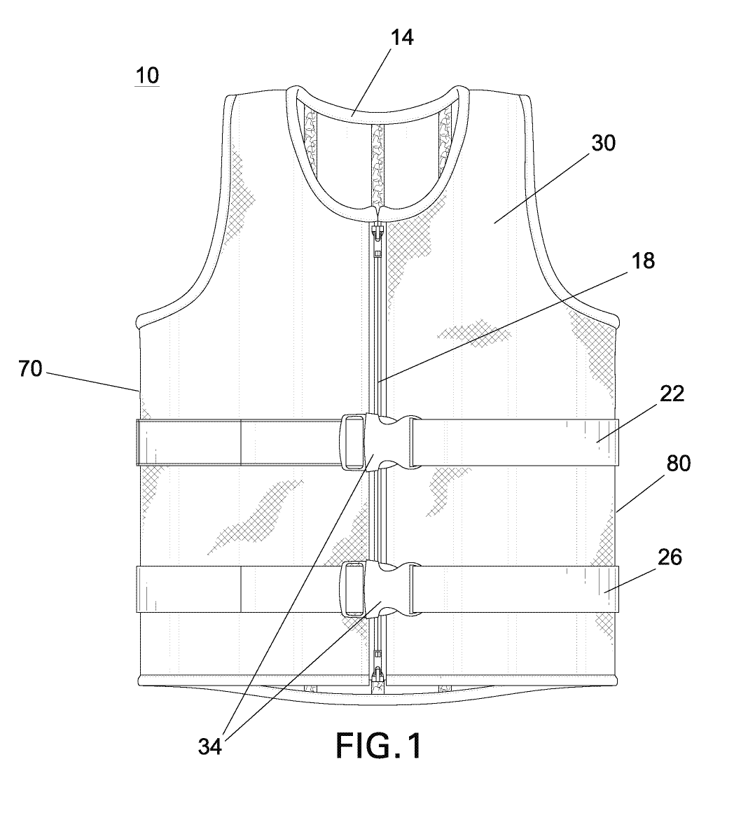

Referring generally to FIGS. 1-4, garment (10) has a front (30), back (50), left side (70), and right side (80). The front (30), back (50), left side (70), and right side (80) each have an interior surface and an exterior surface. The front (30) and back (50) of the garment may be sewn together, or may be joined at the top of the garment using an adjustable attachment to allow the garment to fit users of various sizes. The top of the garment also has a neck hole (14).

The front (30) of garment (10) may have a centrally positioned, vertically oriented opening (18). Opening (18) may extend from neck hole (14) to the bottom edge of the front of the garment. Alternatively, opening (18) may extend from the neck hole to a location between the neck hole and the bottom edge of the front of the garment. Opening (18) may be secured with a zipper as shown in FIG. 1. Alternatively, opening (18) may be secured with snaps, or other means of closure known in the art. Opening (18) may be separated when the garment is put on or taken off the user's body.

The front (30) and/or back (50) of garment (10) may comprise a plurality of panels joined together by attachment means such as a zipper or hook and loop material. The panels may extend from the top of the garment to the bottom of the garment, or may extend circumferentially around the garment (e.g., from the front (30) to the back (50) and around to the front (30) again). Each panel may be detached from the rest of the garment, leaving an exposed edge of the garment. A strip of material that may be, but need not be, the same material from which the rest of the garment is made, may be inserted between a detached panel and an exposed edge of the garment. Thereby, the edge of the strip of material adjacent to the detached panel may have attachment means, such as a corresponding zipper or hook and loop material, that allows the strip of material to attach to the detached panel, and the edge of the strip of material adjacent to the exposed edge of the garment may have attachment means, such as a corresponding zipper or hook and loop material, that allows the strip of material to attach to the exposed edge of the garment. By inserting the strip of material in that way, the garment size may be expanded horizontally to accommodate users with larger chests, or may be extended vertically to accommodate taller users. Also, by reversing the procedure described above, one or more strips of material may be removed from the garment to reduce the size or shorten the garment.

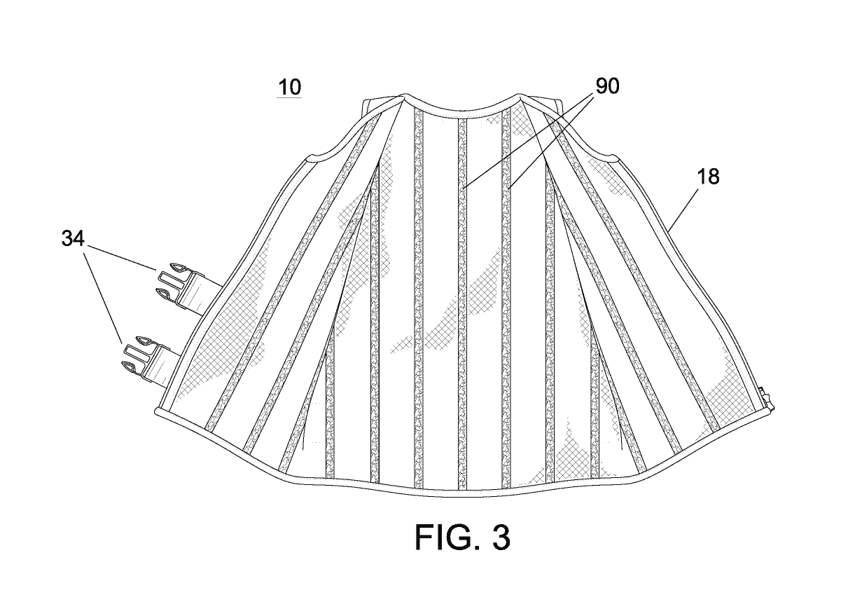

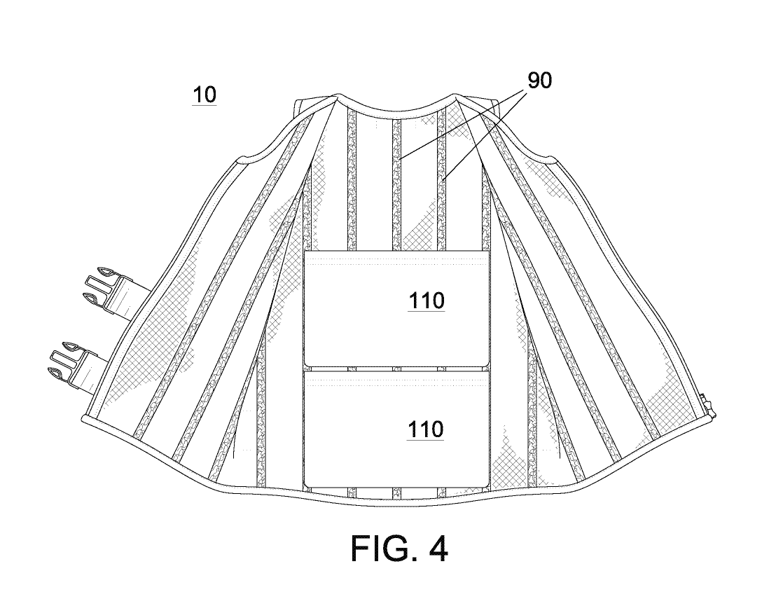

As shown in FIGS. 3 and 4, provided along the interior of the garment may be one or more fastening strips (90) used to removably fasten thermal packets, or envelopes containing thermal packets (110), to garment (10). The fastening strips (90) may comprise one side of a “hook-and-loop” pair (e.g., Velcro®). Preferably fastening strips (90) comprise the loop side of the hook-and-loop pair.

As shown in FIG. 3, each fastening strip (90) may be arranged vertically (parallel to the vertical axis of the garment). Alternatively, each fastening strip (90) may be arranged horizontally (perpendicular to the vertical axis of the garment) or diagonally (arranged along a diagonal line relative to the vertical axis of the garment).

In one embodiment, a first plurality of fastening strips that are parallel to each other may be arranged on the right front side of the interior of the garment, along diagonal lines at a first angle relative to the vertical axis. The first angle may be between 30 degrees and 60 degrees offset from the vertical axis of the garment. A second plurality of fastening strips that are parallel to each other may be arranged on the left front side of the interior of the garment, along diagonal lines at the same angle as the first angle, or at a second angle that is the mirror image of the first angle. The second angle may be between 30 degree and 60 degrees. A third plurality of fastening strips that are parallel to each other may be arranged on the right back side of the interior of the garment along diagonal lines at an angle that is the same as the first angle and/or the second angle relative to the vertical axis. A fourth plurality of fastening strips that are parallel to each other may be arranged on the left back side of the interior of the garment, along diagonal lines at an angle that is the same as the first angle and/or the second angle relative to the vertical axis.

The fastening strips (90) may be evenly spaced apart, and a specific number of fastening strips (90) may be included for a given length of an interior surface of garment (10). For example, three four, five, six, seven, eight, nine, or ten fastening strips (90) may be included per foot of an interior surface of garment (10). Alternatively, all or substantially all of the interior surface of the interior panel of the front (30), back (50), right side (70), and/or left side (80) may comprise the loop side of the hook-and-loop material.

One or more thermal packets may be attached to the fastening strips by removably adhering the corresponding material of a hook-and-loop pair to each thermal packet. For example, one side of each thermal packet may be covered with hook-and-loop material. Alternatively, strips of hook-and-loop material may be adhered to one side of a thermal packet.

In addition or in the alternative, as shown in FIG. 4, one or more envelopes (110) may be used to attach thermal packets to the fastening strips. A thermal packet may be inserted into the envelope through an opening at the top, left side, right, side, or bottom of the envelope. The opening may be sealable using a hook-and-loop closure, zipper, one or more flaps, one or more snaps, or other means of closure. One side of hook-and-loop material may cover the front panel of each envelope. Alternatively, fastening strips comprising one side of hook-and-loop material may be attached to the front panel.

Preferably, the front panel of each envelope (110) may further comprise an insulting material. Insulating the front panel of the envelope would reduce the amount of heat or cold generated from the thermal packet that escapes through the envelope.

In addition or in the alternative, one or more air-tight envelopes may be used to adjust the fit and/or compression of the garment. The opening of each envelope may include and be sealed by an air-tight closure, such as the closure provided on Ziploc® bags. By opening the envelope, separating the sides of the envelope and/or injecting air or another gas, such as nitrogen, into the envelope, and then closing the envelope with an air-tight seal, the envelope may form an inflated bladder that can be attached to the interior of the garment using one of the attachment means described above, including hook and loop material.

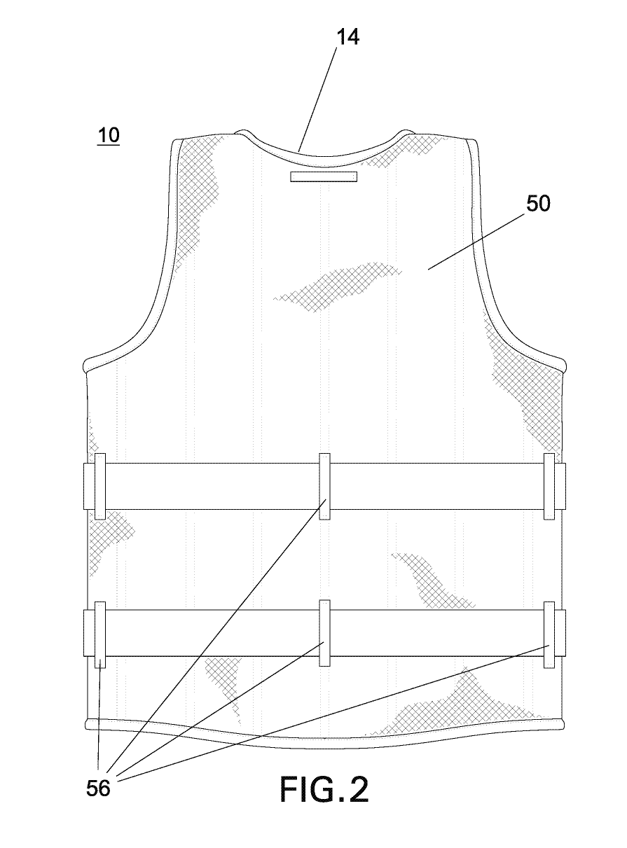

As shown in FIGS. 1-2, the temperature-altering and posture enhancing garment (10) may have one or more pairs of exterior fitting straps. For example, garment (10) may have one or more exterior fitting straps that extend circumferentially around the garment. The embodiment shown in FIGS. 1-4 has two such exterior fitting straps (22, 26). One or more of exterior fitting straps (22, 26) may have a strap adjuster that allows the strap to be tightened to apply compression and encourage proper posture.

As shown in FIG. 2, each exterior fitting strap (22, 26) may be held to garment (10) by, for example, threading the exterior fitting strap (22, 26) through one or more loops (56) attached to garment (10). Alternatively, each exterior fitting strap may be connected to the garment by sewing the strap to the garment. The two ends of each exterior fitting strap (22, 26) may be joined together, preferably at the front (30) of garment (10), using a buckle (34) or other means of fastening. Preferably at least one of the exterior fitting straps is located at a height near the bottom of the user's rib cage, such that it will be placed adjacent to or below the Xiphoid Process of the wearer and adjacent to or above the wearer's eighth rib. One or more of exterior fitting straps (22, 26) may have a strap adjuster that allows the strap to be tightened to apply compression and encourage proper posture.

Alternatively, a first exterior fitting strap may be attached to the back (50) of garment (10) and wrap around the left side (70) of garment (10), and a second exterior fitting strap may be attached to the back (50) of garment (10) and wrap around the right side (70) of garment (10). Those exterior fitting straps may be joined together, preferably at the front (30) of the garment, using a buckle (34) or other means of fastening.

Regardless of how each strap is connected or held to garment (10), preferably at least one exterior fitting strap (22) is located at a height near the bottom of the user's rib cage, such that it will be placed adjacent to or below the Xiphoid Process of the wearer and adjacent to or above the wearer's eighth rib. During activity by the user, or due to the user's girth, one or more exterior fitting straps (22, 26) and/or buckles (34) may be urged higher or lower relative to the torso of the wearer. To ensure that one or more exterior fitting straps (22, 26) and/or buckles (34) remain in a desired position, one or more components may be included on or used with garment (10). For example, one or more loops (56) may be attached to garment (10) adjacent to opening (18), and/or substantially adjacent to opening (18). For one or both exterior straps (22, 26), a loop (56) may be included on either side of opening (18). Threading one or both exterior straps (22, 26) through loops near opening (18) may keep the buckles at a desired location relative to the wearer's torso.

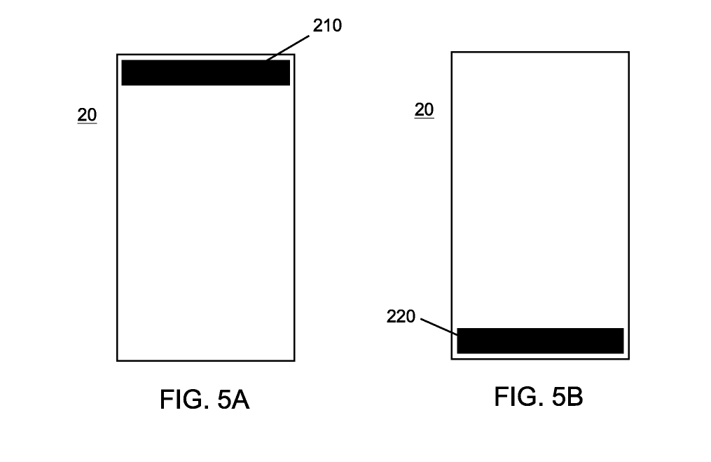

In addition, or in the alternative, a fastening band may be used to keep exterior fitting straps (22, 26) and/or buckles (34) at or near a desired position. FIGS. 5A and 5B show a front view and back view, respectively, of exemplary fastening band (20). The fastening band may be made from a flexible material that may be elastic. The fastening band may be made from neoprene, nylon, rubber, or another suitable material. The fastening band may also be made from more than one material. For example, part of the fastening band may be made from a flexible material, while another portion of the band may be made from an inflexible material.

The fastening band may include one or more means for securing the ends of fastening band together. For example, the front side of fastening band (20) shown in FIG. 5A includes one side of a “hook-and-loop” pair (210), and the back side of fastening band (20), shown in FIG. 5B, includes a corresponding side of a “hook-and-loop” pair (220). In the alternative, both hook-and-loop pairs of material may be located on the same side of the fastening band. Fastening band (20) may be wound around exterior fitting straps (22, 26) and/or buckles (34), and the two sides of the “hook-and-loop” pair (210, 220) may be joined together to form fastening band (20) into a loop. In addition or in the alternative, other means of joining the two end of fastening band (20) may be included at either end of fastening band (20), including, snaps or a zipper.

Another component that may be used to ensure that one or more exterior fitting straps (22, 26) and/or buckles (34) remain in a desired position is a strap or line having two ends, wherein both ends may be connected to exterior fitting straps (22, 26) and/or buckles (34), or one end may be attached to garment (10) and the second end may be connected to an exterior fitting strap (22, 26) and/or buckle (34). For example, a nylon rope having a length that is the same or approximately the same as the desired distance between two exterior fitting straps (22, 26) and/or buckles (34) may include a clasp or clip on either end of the rope. Each clasp or clip may be removably or permanently attached to an exterior fitting strap (22, 26) and/or buckle (34). The strap or line may also be attached to garment (10) to help prevent exterior fitting straps (22, 26) and/or buckles (34) from moving relative to the wearer's torso.

In addition to the elements and features described above, or in the alternative, the temperature-altering and posture enhancing garment (10) may have a belt that provides lumbar support. The belt may have padding along the back and may be three to seven inches wide. The belt may be situated near the bottom of the garment to rest at the user's lower back.

In addition or in the alternative, the temperature-altering and posture enhancing garment (10) may have one or more cinch cords. The cinch cords would be preferably located throughout the garment to prevent the garment from sliding upward along the user's torso while the user moves.

In addition or in the alternative, the temperature-altering and posture enhancing garment (10) may have one or more interior fitting straps. One end of each interior fitting strap may be attached to the interior of the top of the garment. Each interior fitting strap may be arranged vertically along the interior of the front of the garment and attached to a separate garment being worn on the lower half of the wearer's body, for example, by attaching to a belt loop of the user's pants, or other means of attachment. A strap adjuster may be fitted to each interior fitting strap to allow the user to tighten the strap. The interior fitting straps may be crossed across the wearer's torso if desired.

In addition or in the alternative, the temperature-altering and posture enhancing garment (10) may have boning on its interior or exterior to further ensure proper posture and make the garment more rigid.

The temperature-altering and posture enhancing garment (10) may have one or more stimulation devices. The stimulation device may comprise battery powered components that vibrate. The vibration may be used to further soothe muscles. Controls such as touch sensitive buttons may be included on garment (10) to set the vibration to be provided in pulses or continuously, and to adjust the intensity of the vibration.

The temperature-altering and posture enhancing garment (10) may have a processing unit to monitor and/or control the one or more stimulation devices or other components incorporated in the garment. For example, one or more biometric sensors may be placed in the garment to monitor body temperature, heart rate, blood glycolysis or other conditions. Other sensors such as a pedometer, an altimeter, a barometer, a GPS unit, a gyroscope, and an accelerometer may also be included to record other measurements such as body position, steps taken, and distance traveled.

The processing unit may receive data from each sensor. The data may be stored in a memory unit incorporated in the garment, and/or may be transmitted to another device such as a cellular phone or a laptop computer.

Feedback may be presented to the wearer based on the data collected. The garment may include a light or sound emitting device, or a haptic device to provide feedback. For example, sensors in the garment may assess the user's posture based on the position of the garment. To the extent that the user's posture deviates from a predetermined position, a light or sound emitting device or a haptic device may be activated to alert the user.

While the invention has been described with reference to the preferred embodiment and alternative embodiments, which embodiments have been set forth in considerable detail for the purposes of making a complete disclosure of the invention, such embodiments are merely exemplary and are not intended to be limiting or represent an exhaustive enumeration of all aspects of the invention. Further, it will be apparent to those of skill in the art that numerous changes may be made in such details without departing from the spirit and the principles of the invention. It should be appreciated that the invention is capable of being embodied in other forms without departing from its essential characteristics.

What is claimed is:

1. An apparatus for altering the temperature of select areas of the trunk of the human body and enhancing posture, comprising:
a garment having a front side, back side, left side, and right side arranged about a vertical axis, each side having an interior panel and an exterior panel;
a plurality of fastening strips attached to the interior of the garment, arranged parallel to each other;
an envelope removably attachable to a fastening strip, said envelope having a sealable opening at the top of the envelope;
a first exterior fitting strap having a first end and a second end, wherein the first end and the second end are releasably joined together by a first buckle at the front of the garment, and wherein the first exterior fitting strap is adjacent to or below the Xiphoid Process of a wearer of the garment and adjacent to or above the eighth rib of the wearer; and
a second exterior fitting strap having a first end and a second end, wherein the first end and the second end are releasably joined together by a second buckle at the front of the garment;
wherein tightening the first exterior fitting strap and the second exterior fitting strap promotes proper posture.

2. The apparatus of claim 1 wherein the opening at the top of the envelope is sealable with a zipper.

3. The apparatus of claim 1 wherein the opening at the top of the envelope is sealable with hook-and-loop material.

4. The apparatus of claim 1 wherein the envelope forms an air-tight enclosure when sealed.

5. The apparatus of claim 1 wherein one side of the envelope is covered by hook-and-loop material.

6. The apparatus of claim 1 wherein one side of the envelope comprises fastening strips joinable to a plurality of fastening strips attached to the interior of the garment.

7. The apparatus of claim 1 further comprising a thermal packet inserted within the envelope.

8. The apparatus of claim 1, wherein the plurality of fastening strips are arranged along a diagonal line relative to the vertical axis of the garment.

9. The apparatus of claim 1, wherein the plurality of fastening strips comprise:
a first set of fastening strips attached to the interior of the garment on the right front side of the garment, positioned parallel to each other and arranged along a diagonal line relative to the vertical axis of the garment;
a second set of fastening strips attached to the interior of the garment on the left front side of the garment, positioned parallel to each other and arranged along a diagonal line relative to the vertical axis of the garment, and arranged at an angle that is a mirror image of the first set of fastening strips;
a third set of fastening strips attached to the interior of the garment on the right half of the back of the garment, positioned parallel to each other and arranged along a diagonal line relative to the vertical axis of the garment;
a fourth set of fastening strips attached to the interior of the garment on the left half of the back of the garment, positioned parallel to each other and arranged along a diagonal line relative to the vertical axis of the garment, and arranged at an angle that is a mirror image of the third set of fastening strips.

10. The apparatus of claim 1, further comprising a fastening band having a first end and a second end, wherein the fastening band is wound around the first buckle and the second buckle.

11. The apparatus of claim 10, wherein the first end of the fastening band comprises hook-and-loop material, and the second end of the fastening band comprises hook-and-loop material.

12. An apparatus for altering the temperature of select areas of the trunk of the human body and enhancing posture, comprising:
a garment having a front side, back side, left side, and right side arranged about a vertical axis, each side having an interior panel and an exterior panel, wherein the interior surface of one of the interior panels is covered with one side of hook-and-loop material;
an envelope removably attachable to the hook-and-loop material, said envelope having a sealable opening at the top of the envelope;
a first exterior fitting strap having a first end and a second end, wherein the first end and the second end are releasably joined together by a buckle at the front of the garment, and wherein the first exterior fitting strap is adjacent to or below the Xiphoid Process of a wearer of the garment and adjacent to or above the eighth rib of the wearer; and
a second exterior fitting strap having a first end and a second end, wherein the first end and the second end are releasably joined together by a buckle at the front of the garment;
wherein tightening the first exterior fitting strap and the second exterior fitting strap promotes proper posture.

13. The apparatus of claim 12 wherein the opening at the top of the envelope is sealable with a zipper.

14. The apparatus of claim 12 wherein the opening at the top of the envelope is sealable with hook-and-loop material.

15. The apparatus of claim 12 wherein the envelope forms an air-tight enclosure when sealed.

16. The apparatus of claim 12 wherein one side of the envelope is covered by hook-and-loop material.

17. The apparatus of claim 12 wherein one side of the envelope comprises fastening strips joinable to the hook-and-loop material.

18. The apparatus of claim 12 further comprising a thermal packet inserted within the envelope.

19. The apparatus of claim 12, further comprising a fastening band having a first end and a second end, wherein the fastening band is wound around the first buckle and the second buckle.

20. The apparatus of claim 19, wherein the first end of the fastening band comprises hook-and-loop material, and the second end of the fastening band comprises hook-and-loop material.
